(19) United States
(12) Patent Application Publication
(10) Pub. No.: US 2018/0256788 A1
(43) Pub. Date: Sep. 13, 2018

(54) SYSTEM FOR WOUND ASPIRATION AND BINDING DEVICE

(71) Applicant: PFM MEDICAL AG, Koeln (DE)

(72) Inventors: Daniel BACKES, Gusenburg (DE); Christian BAUMEISTER, Trier (DE)

(21) Appl. No.: 15/760,909

(22) PCT Filed: Jul. 6, 2016
(86) PCT No.: PCT/EP2016/065921

§ 371 Date: Mar. 16, 2018

(30) Foreign Application Priority Data
Sep. 17, 2015 (DE) 20 2015 104 952.8

(51) Int. Cl. A61M 1/00; B65D 63/10; F16M 13/02; F16B 2/08

(52) U.S. Cl. CPC A61M 1/0001; B65D 63/1027; F16M 13/022; A61M 2209/084; A61M 2207/00; A61M 2209/082; F16B 2/08

(57) ABSTRACT

The invention relates to a system for wound aspiration, comprising: at least one vacuum vessel with an opening for suspending or securing the vacuum vessel, and a binding device for securing the vacuum vessel on a holder, preferably for suspending and/or securing the vacuum vessel on a stand, with a band-shaped main part, wherein the main part has profiled thickenings, which are arranged at regular intervals, and a lug, which is formed substantially at the end of the main part and which serves to receive and to releasably fix at least one of the profiled thickenings, characterized in that the main part has at least one fixing element for fixing the binding device in the opening of the vacuum vessel.

### FIELD

The invention relates to a system for wound aspiration, comprising at least one vacuum vessel with at least one opening for suspending and/or securing the vacuum vessel on a holder, preferably for suspending and/or securing the vacuum vessel on a stand, with a band-shaped main part, wherein the main part has profiled thickenings, which are arranged at regular intervals, and a lug, which is formed substantially at the end of the main part and which serves to receive and to releasably fix at least one of the profiled thickenings, characterized in that the main part has at least one fixing element for fixing the binding device in the opening of the vacuum vessel.

Furthermore, the invention relates to a binding device.

### BACKGROUND

A lug in the sense of the present invention is an opening or the like, through which at least a part of the binding device, in particular the profiled thickenings arranged at regular intervals on the main part, can be introduced and into which at least part of the binding device can be received for retraction. In this case, the lug advantageously has two areas. The first area is formed so wide that pulling in or pushing through at least a part, in particular of the profiled thickenings, of the binding device is possible immediately adjoining the first area of the lug is the second area of the lug, which is tapered in relation to the first area, so that a pushing through or pulling in of the profiled thickenings is not possible, but the area between the profiled thickenings intervene in the tapering. Thus, the second area of the lug allows locking of the profiled thickenings within the lug of the binding device.

From the prior art a binding device is known, according to which one of the profiled thickenings can be locked in a lug for receiving and releasably fixing the profiled thickenings. In this way the known binding device forms a closure device when closing, with which for example a closure of sacks and/or bags is possible. The arrangement of several profiled thickenings can be adapted to the type of objects to be closed. Furthermore, it is known to form the binding device into a kind of loop by introducing one of the profiled thickenings into the formed lug for receiving and releasably fixing the profiled thickenings. For example, an opening of an article can be arranged on the thus formed loop, so that the object can be suspended or fastened by means of the binding device.

By way of example, it is known from the prior art to use such a binding device to suspend or attach systems for wound aspiration or wound drainage to a stand or infusion stand. A system for wound aspiration is also referred to as so-called redon drainage. In this case, a drainage in the form of a tube with a perforation is left in the surgical wound after a successful operation. The tube is connected to a container which is provided with a negative pressure. The negative pressure creates a suction effect on the perforation of the tube, which aspirates wound secretions arising from the wound via the tube into the container.

For easy handling of a system for wound aspiration the vacuum vessel is usually attached via an opening by means of a binding device to an IV stand or stand.

A disadvantage of the known binding device and the known systems for wound aspiration is that during the production the mounting of the binding device must be done manually within an opening of the vacuum vessel by locking of one the profiled thickenings of the binding device in the lug for receiving and releasably fixing at least one of the profiled thickenings, since otherwise the binding device could fall out of the opening.

Furthermore, in attempts to automate the assembly of the binding device, it has been found that mere insertion of the known binding device is not sufficient, since without locking one of the profiled thickenings in the opening the binding device tends to slip out from the opening of the vacuum vessel after the successful insertion. This is due in particular to the fact that the opening is larger than the widest point of the cross section of the binding device. An insertion is only sufficient if the binding device is inserted so far into the opening, that the widest point of the lug is not pushed through the opening and thus rests against the opening of the object to be fastened. The problem with automated assembly is therefore further that by the flexible design of the binding device it must be inserted so far through the opening that it does not “fall out” again from the opening to be fastened. For this purpose, however, the binding device would have to be held at the “remote” end with respect to the opening of the object to be fastened. However, due to the flexibility of the binding device, insertion as described above is very difficult.

Based on this prior art, the object of the invention is to avoid the disadvantages known from the prior art and to provide a system with an improved binding device.

For the technical solution of this object, the invention proposes a system with a binding device, which characterized in that the main part has at least one fixing element for fixing the binding device in an opening of the object to be fastened, preferably an opening of the vacuum vessel.

### SUMMARY

The invention is based on the finding that during the manufacturing of a system for wound aspiration the mounting of a binding device for fastening an object to a holder, in particular for suspension and/or attachment of a vacuum vessel to a stand, can be considerably simplified by providing the binding device additionally with at least one fixing element. The at least one fixing element allows the binding device to be inserted only through the opening of the object to be fastened, wherein the at least one fixing element now prevents the binding device from being pulled out or slipping out of the opening. In particular, this includes a pulling out or slipping out of the binding device counter to the inserting direction. A locking of one of the profiled thickenings in the lug of the binding device is therefore no longer necessary during the production, or a cumbersome wide insertion in to the opening of the object to be fastened.

An embodiment of the invention provides that the at least one fixing element is arranged directly adjacent to or adjacent to the end comprising the lug. This has the advantage that during fixing one of the profiled thickenings of the binding device in the lug of the binding device, the handling during attachment of an object to the holder is not adversely affected by the at least one fixing element.

In a further embodiment, the at least one fixing element is arranged on the side facing away from the profiled thickened side. The at least one fixing element is thus arranged at the end of the band-shaped main part, more precisely, the at least one fixing element is arranged at the end and at the opposite end of the profiled thickenings of the binding device.

In a preferred embodiment, the at least one fixing element is arranged between the profiled thickenings and the end formed lug. This has the advantage that the handling of the binding device is improved, in particular after fixation of the binding device with an opening of an object to be fastened, since in this way the lug of the binding device protrudes out of the opening of the vacuum vessel on the opposite from the profiled thickenings of the binding device, and thus an insertion and fixation of at least one of the profiled thickenings within the lug of the binding device is decidedly facilitated in case of a releasable fixation.

A further preferred embodiment of the invention provides that the binding device is characterized by a stop element which is arranged immediately adjacent to or adjacent to the at least one fixing element and prevents pushing of the binding device through the opening of the object to be fastened. Accordingly, the binding device is fixed, for example, after insertion into the opening of the vacuum container in this. The at least one fixing element and the stop element then surround the opening of the vacuum vessel so that the opening is enclosed by the at least one fixing element and the stop element. In this case, the stop element prevents a pulling out or slipping of the binding device, in particular in the direction of insertion of the binding device in the opening of the vacuum vessel, since the widest point of the binding device, formed by the stop element, is larger than the diameter of the opening.

An embodiment of the invention provides that the at least one fixing element is designed in the form of a barb. The widest point of the barb is wider or larger than the opening of the object to be fastened. This can optimize the fixation in the opening of a vacuum vessel, since a pulling out or slipping out of the fixing element is completely prevented. Only by breaking or bending the barb it is possible to remove the at least one fixing element against the direction of action of the barb from an opening, preferably the opening of the vacuum vessel.

A further embodiment of the invention provides that the at least one fixing element is designed substantially oval-shaped or oval. The design of the at least one fixing element similar to an oval can prevent the at least one fixing element from getting caught on objects in the working or assembly environment during use.

In a further embodiment of the invention, the at least one fixing element and/or the stop element is formed from a solid profiled thickening of the main part. This is advantageous for the production of the binding device. For example, corresponding cavities can be produced in the injection mold, so that a binding device produced by injection molding is provided with the inventive at least one fixing element and/or stop element.

Furthermore, an embodiment of the invention provides that the at least one fixing element has at least one material recess in the main part. On the one hand, the manufacturing costs of the binding device according to the invention can be reduced by material savings, on the other hand, the material strength of the at least one fixing element is changed, so that, for example, a deformation of the at least one fixing element is made possible. Thus, the binding device according to the invention is easier to handle, since for example the introduction and/or retraction of the at least one fixing element in or out of an opening of an object to be fastened, preferably an opening of the vacuum vessel, is facilitated.

In an advantageous embodiment, the binding device according to the invention is made of a bendable or flexible material. In particular, the binding device according to the invention is made of polyethylene or polypropylene or of a combination of these materials.

An advantageous embodiment of the invention provides that the binding device is formed in one piece.

In a further embodiment, the at least one fixing element and the lug are integrally formed. In this case, for example, the barb can be attached directly to the lug or the lug is designed such that the shape simultaneously fulfils the function of an oval fixing element.

The object underlying the invention is achieved by a system for wound aspiration, which comprises at least one vacuum vessel with an opening for attachment or suspension of the vacuum vessel, and a binding device.

The present invention further comprises:

A method of producing a wound aspiration system,

characterized by:

connecting the vacuum vessel and the binding device, wherein the binding device is mounted automatically, preferably by means of a gripper.

During the production of an inventively designed system for wound aspiration an automatic and/or mechanical assembly of the binding device in an opening for fastening the vacuum vessel to a stand or the like can be carried out. In general, the automatic and/or mechanical assembly of slim and flexible components, like for example the binding device according to the invention, is difficult because corresponding assembly robots can badly grasp and handle such components. In addition, a locking of one of the profiled thickenings of the binding device in the lug for receiving such thickening is a very complex movement for a mounting robot. Consequently, hitherto, manual assembly by humans has usually been performed to achieve the desired result.

The invention further comprises:

A method, characterized in that during connecting a gripper grips one end of the binding device and introduces it into an opening of an object to be fastened, so that the binding device is fixed to the object.

According to one aspect of the invention, it is provided that the binding device according to the invention is inserted, for example, in the opening of the vacuum vessel or of the object to be fastened. By the at least one fixing element or by the at least one fixing element and the stop element the binding device is fixed by merely insertion into the opening. This assembly process can be easily automated, since the above mentioned insertion and locking of the binding device is no longer necessary and slipping out is no longer possible.

In addition, the invention comprises:

A method, characterized in that the at least one stop element formed on the binding device prevents a pushing through of the binding device through the opening.

### BRIEF DESCRIPTION OF THE DRAWINGS

Further details, features and advantages of the invention will be explained in more detail with reference to the embodiments illustrated in the figures. It shows:

FIG. 1 is an embodiment of a binding device according to the invention;

FIG. 2a and FIG. 2b are embodiments of a system for wound aspiration according to the invention,

FIG. 3a through FIG. 3d are embodiments of fixing elements of a binding device, and

FIG. 4 is an embodiment of a binding device according to the invention comprising a fixing element and a stop element.

### DETAILED DESCRIPTION

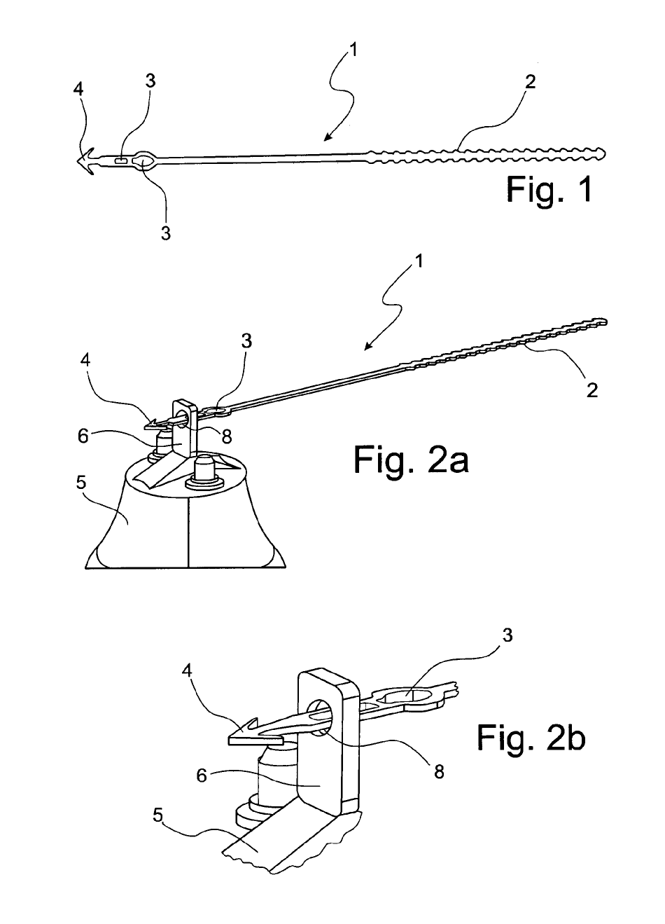

FIG. 1 shows a schematic view of a binding device 1 according to the invention for suspending and/or securing of a vacuum vessel 5, not shown in FIG. 1, on a not shown stand or IV stand. The binding device 1 with a band-shaped main part comprises profiled thickenings 2, which are arranged at regular intervals at the end of the binding device 1. At the opposite end of the profiled thickenings 2 of the main part of the binding device 1 are presently two immediately adjacent arranged lugs 3 for receiving and releasable fixation of the thickenings 2. In case for locking the binding device 1, respective thickenings 2 can engage in one of the lugs 3 at the same time. The closing effect of the binding device is thereby enforced. Next to the lugs 3 a fixing element 4 is arranged in the form of a barb. The fixing element 4 is used for fixing the binding device 1 in an opening or the like of a not shown object.

FIG. 2a shows a binding device 1 according to the invention mounted on a vacuum vessel 5. The binding device 1 according to the invention and the vacuum vessel 5 together form a system for wound aspiration. The cylindrical vacuum vessel 5 has on its upper side an opening 6 which is provided with an attachment hole. Through the attachment opening 8 of the opening 6 the binding device is inserted. In this case, the barb 4 of the binding device 1 is inserted through the attachment opening 8 of the opening 6 in such a way that a withdrawal or slipping out of the binding device 1 from the attachment opening 8 of the opening 6 is prevented.

FIG. 2b shows a partial enlargement of the connecting region of the binding device 1 and the opening 6 of the vacuum vessel 5. In this case, the binding device 1 is inserted into the opening 6 such that the barb 4 is located on one side of the opening 6 and the lug 3 for receiving and releasably fixing at least one of the profiled thickenings 2 on the opposite side of the opening 6. In the event that the attachment hole of the opening 6 is smaller than the widest point of the lug 3 of the binding device, the lug 3 simultaneously forms a stop element 7, which prevents the binding device against the blocking action of the barb from being pulled out or slipping out of the attachment opening 8 of the opening 6.

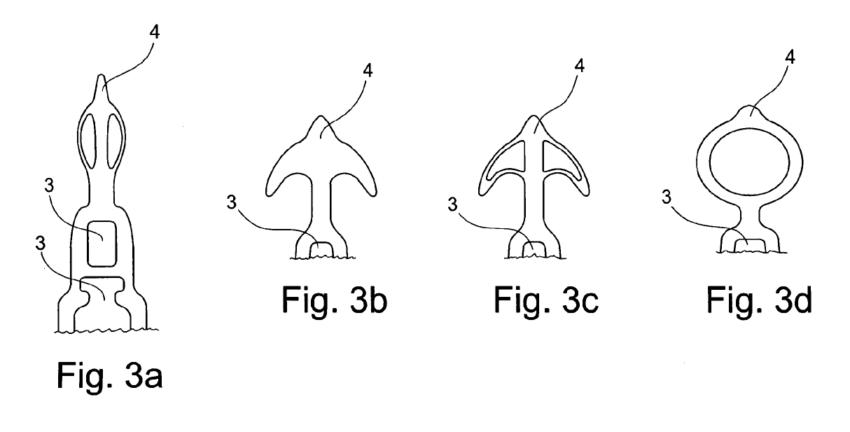

In FIGS. 3a to 3d different embodiments of a respective fixing element 4 according to the invention are shown. Thus, FIG. 3a shows a fixing element 4, which is formed oval. In addition, the fixing element has recesses. These serve on the one hand to reduce the material of the binding device 1, and on the other hand allow the material recesses flexibility of the fixing element 4 to a certain extent. This facilitates for example the insertion or retraction of a binding device 1 in an opening 6 of a vacuum vessel 5, since the fixing element 4 is deformable thereby. Compared to an embodiment of a fixing element 4 in the form of a barb, as shown in FIG. 3b, a substantially oval-shaped configuration of a fixing element 4 facilitates the use as a snagging on other objects, for example during assembly of a system for wound aspiration according to the invention, is minimized.

In comparison to FIG. 3b the fixing element 4 in the form of a barb according to FIG. 3c has recesses and is therefore not solid. The above mentioned advantages of the material recesses of the fixing element 4 are also true for the fixing element 4 in the form of a barb as shown in FIG. 3c.

In FIG. 3d a fixing element 4 is shown, which has an oval basic shape. In contrast to the fixing element 4 according to FIG. 3a, which also has an oval basic shape, the fixing element 4 according to FIG. 3d is completely recessed, so that only the outer edge in the form of a ring forms the fixing element 4. This further increases the flexibility of the fixing element. In addition, less material is needed in the production.

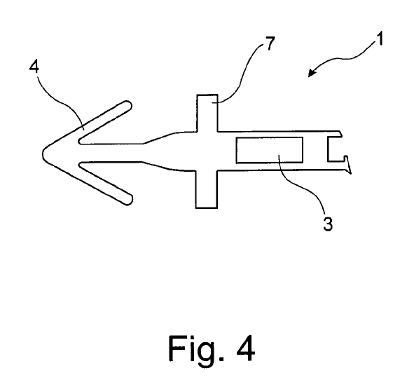

FIG. 4 shows a partial view of an embodiment of a binding device 1 according to the invention. The binding device 1 has a stop element 7 in addition to the barb 4. The stop element 7 is arranged directly adjacent or adjacent to the barb 4 and is provided to prevent a pushing through an opening 6 of a not shown vacuum vessel 5. The stop element 7 is formed in its width such that the width of the stop element 7 is greater than the diameter of a mounting 8 or the like opening 6 of a vacuum vessel 5 or an object to be fastened with a corresponding opening, so that the stop element 7 and thus also the binding device 1 is not movable through the attachment opening 8.

The exemplary embodiments illustrated and described in the figures merely serve to explain the invention and are not restrictive of it.

### LIST OF REFERENCE NUMBERS

1 binding device
2 profiled thickenings
3 lug for receiving and releasably fixing the thickenings
4 fixing element
5 vacuum vessel
6 opening with hole for securing or suspending the vacuum vessel
7 stop element
8 mounting opening of the tab

What is claimed is:

1. A system for wound aspiration, comprising:
at least one vacuum vessel with an opening for suspending or securing the vacuum vessel; and
a binding device for securing the vacuum vessel on a holder, preferably for suspending and/or securing the vacuum vessel on a stand, with a band-shaped main part, wherein the main part has profiled thickenings, which are arranged at regular intervals, and a lug, which is formed substantially at the end of the main part and which serves to receive and to releasably fix at least one of the profiled thickenings,
wherein
the main part has at least one fixing element for fixing the binding device in the opening of the vacuum vessel.

2. The system according to claim 1, wherein the at least one fixing element is arranged adjacent to the end comprising the lug.

3. The system according to claim 2, wherein the at least one fixing element is arranged on the side facing away from the profiled thickened side.

4. The system according to claim 2, wherein the at least one fixing element is arranged between the profiled thickenings and the end comprising the lug.

5. The system according to claim 1, comprising a stop element, which is arranged immediately adjacent to or adjacent to the at least one fixing element and prevents pushing of the binding device through the opening of the object to be fastened, preferably the opening of the vacuum vessel.

6. The system according to claim 1, wherein the at least one fixing element is designed in the form of a barb.

7. The system according to claim 1, wherein the at least one fixing element is designed substantially oval-shaped.

8. The system according to claim 5, wherein the at least one fixing element and/or the stop element is formed from a solid profiled thickening of the main part.

9. The system according to claim 6, wherein the at least one fixing element has at least one material recess in the main part.

10. The system according to claim 1, wherein the binding device is made of a flexible material.

11. The system according to claim 1, wherein the binding device is formed in one piece.

12. The system according to claim 1, wherein the at least one fixing element and the lug are integrally formed.

13. A binding device, wherein the binding device is designed for use with the system according to claim 1.

14. A method of producing a wound aspiration system according to claim 1, characterized by connecting the vacuum vessel and the binding device, wherein the binding device is mounted automatically, preferably by means of a gripper.

15. The method according to claim 14, wherein during connecting a gripper grips one end of the binding device and introduces it into an opening of an object to be fastened, so the binding device is fixed to the object.

16. The method according to claim 14, wherein the at least one stop element formed on the binding device prevents a pushing through of the binding device through the opening.
